(19) United States
(12) Patent Application Publication
(10) Pub. No.: US 2008/0091167 A1
(43) Pub. Date: Apr. 17, 2008

(54) Method and Apparatus for Intervertebral Disc Expansion

(75) Inventor: Hai H. Trieu, Cordova, TN (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/924,026

(22) Filed: Oct. 25, 2007

Related U.S. Application Data
(62) Division of application No. 10/314,396, filed on Dec. 7, 2002.

(51) Int. Cl. A61M 31/00

(52) U.S. Cl. 604/509

(57) ABSTRACT

An intervertebral disc is expanded and injected by forming and dilating an opening in the disc annulus and introducing an inflatable member into the disc nucleus pulposus. The inflatable member location within the nucleus pulposus is verified and the inflatable member is gradually inflated for augmenting a space in the nucleus pulposus. The internal pressure and expansion of the inflatable member are monitored. The inflatable member is subsequently deflated and a biomaterial is injected into the augmented space.

### PRIORITY

The present application is a divisional application of application Ser. No. 10/314,396 filed Dec. 7, 2002 entitled “Method and Apparatus for Intervertebral Disc Expansion.”

### BACKGROUND

The present disclosure relates to surgical apparatus and methods, and more particularly to the treatment of intervertebral discs.

This application relates to co-pending U.S. patent application Ser. No. 10/245,955, filed on Sep. 18, 2002, entitled “Collagen-Based Materials And Methods For Augmenting Intervertebral Discs,” naming Hai Trieu and Michael Sherman as inventors. The co-pending application is incorporated herein by reference in its entirety, and is assigned to the assignee of this application.

Degenerated disc disease (DDD) leads to disc dehydration (black disc), gradual collapse, and ultimately leg and/or back pain. Interbody fusion is the current standard of care for DDD. It is desirable that this end-stage treatment be delayed as long as possible by early intervention with less invasive approaches. Disc augmentation by injection of a biomaterial into the disc space has been proposed previously as an early minimally invasive treatment for a degenerated disc. Depending on the level of dehydration and collapse, injection of a biomaterial into the disc space of an intact disc (uncompromised annulus with no significant tears and original nucleus pulposus still in place) may require a high injection pressure and the injectable volume of biomaterial may be limited. High injection pressure increases the overall risk of the procedure including leakage, disc rupture, etc. Limited injectable volume reduces the effectiveness of the treatment and may require multiple treatments to achieve desirable results.

In known methods for intervertebral disc expansion, a cut is made in the disc annulus and disc tissue is removed to provide a passage for the insertion of an expansion device, an expansion material, or both. Also, the nucleus pulposus is removed and replaced by the expansion material and/or expansion device. Furthermore, degeneration of the disc is accelerated when an opening is cut into the disc annulus and tissue is removed.

Therefore, what is needed is a device and method for accessing the nucleus pulposus for expansion of the disc such that no portion of the disc annulus and the nucleus pulposus are removed. Also, what is needed is an apparatus and method for a minimally invasive disc treatment which increases injectable volume at a lower pressure.

### SUMMARY

One embodiment, accordingly, includes an expandable device for intervertebral disc expansion by means of an inflatable member insertable into a dilated opening in an intact intervertebral disc annulus and into a nucleus pulposus of the disc. An inflation device is connected to controllably inflate the inflatable member within the nucleus pulposus without removing the nucleus pulposus.

A principal advantage of this embodiment is that it enables disc expansion with a percutaneous or minimally invasive approach. The disc expansion enables a larger volume of biomaterial injection per treatment. A larger volume of biomaterial injection reduces the number of treatments to achieve desirable level of augmentation. This treatment enables disc expansion without removal of the nucleus pulposus and helps determine the appropriate biomaterial volume prior to injection. Over-injection of the disc, and resulting pain and complications, can be minimized using the proposed device and method. Another advantage is that the disc remains intact such that no portion of the disc annulus or disc nucleus is removed.

### BRIEF DESCRIPTION OF THE DRAWINGS

FIG. 1 is a cross-sectional view illustrating an embodiment of a disc structure.

FIGS. 2A-2F are cross-sectional views illustrating an embodiment of a disc expansion method and apparatus.

FIG. 3 is a cross-sectional view illustrating another embodiment of a disc expansion method and apparatus.

FIG. 4 is a cross-sectional view illustrating another embodiment of a disc expansion method and apparatus.

FIGS. 5A-5D are cross-sectional views illustrating another embodiment of a disc expansion method and apparatus.

### DETAILED DESCRIPTION

A disc structure 10, FIG. 1, generally comprises adjacent vertebrae 12 and 14 of the cervical, thoracic, or lumbar regions of the spine. An intervertebral disc 16 facilitates motion between the vertebrae 12 and 14 while absorbing shock and distributing loads. The disc 16 generally comprises a soft central core, i.e. the nucleus pulposus 18 (disc nucleus), that bears the majority of the load in a healthy disc, and a tough outer ring, i.e. the annulus fibrosis 20 (disc annulus), that surrounds and stabilizes the disc nucleus 18. A pair of cartilage endplates 22 are between each respective vertebrae 12 and 14, and the disc nucleus.

The method and apparatus are used following a patient diagnosis and selection for treatment, and in addition, a discogram to ensure disc annulus integrity.

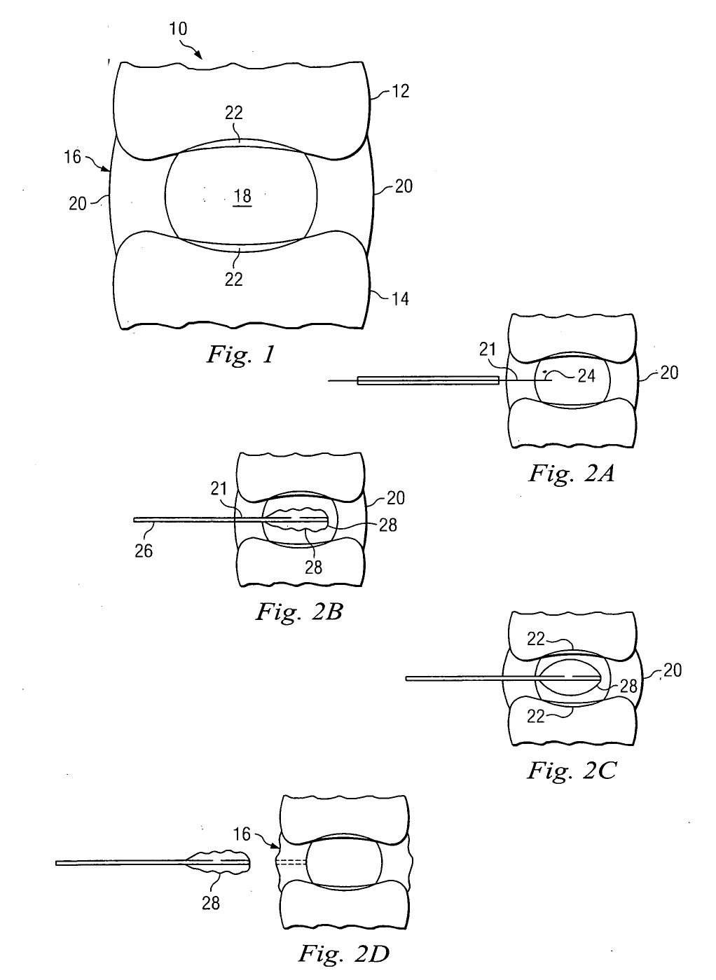

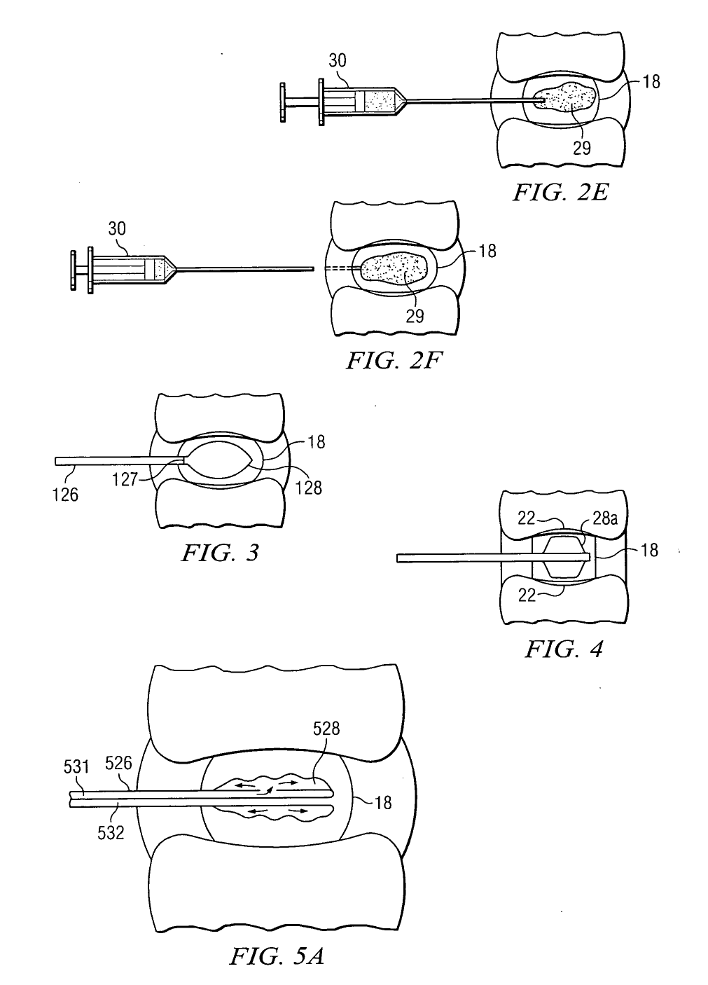

The disc annulus 20, FIG. 2A, is punctured at 21 using a small diameter needle 24. A preferable needle size is 20 gauge. A small diameter (i.e. 1 to 3 mm) high-pressure balloon catheter 26, FIG. 2B, is introduced through the puncture 21 in the disc annulus 20. The location of a balloon 28 attached to catheter 26, in the disc nucleus 18 may be verified using fluoroscopy. The puncture required for insertion of devices for disc expansion and injection is small enough i.e. no greater than 3 mm, that the puncture may completely close, or close sufficiently that the injected biomaterial will remain captured. In the case of a biomaterial that sets up in the disc space after injection, capture of the injected biomaterial is assured. The use of an annulus closure device such as a plug or material such as a sealant is optional.

The balloon 28, FIG. 2C is gradually inflated with a saline and/or radiographic contrast medium such as sodium diatrizoate solution sold under the trademark Hypaque®, while monitoring the internal balloon pressure with a well known pressure gauge. Expansion of the balloon 28 is monitored using fluoroscopy. The rate of inflation and the pattern, size or shape of the balloon 28 can be varied between patients depending on disc condition. As the intradiscal pressure is increased and/or the endplates 22 are spread apart by the balloon 28, the disc annulus 20 is expected to stretch, as it is a viscoelastic material. The balloon may remain inflated from about 1 minute to about 1 hour, which may be varied for each patient. If significant expansion is required, the balloon may remain inflated up to 4 hours or it may be left in the disc space as a temporary implant up to 10 weeks.

As the balloon 28, FIG. 2D, is deflated, the disc 16 becomes slack with an augmented space and reduced intradiscal pressure. Injectable biomaterial 29 such as a collagen gel can be delivered to the disc nucleus 18, FIG. 2E, either through the same catheter, or a different needle 30 may be used after the balloon catheter 26 is deflated and removed. If the same catheter is used for injection, the injection can be done simultaneously as the balloon 28 is being deflated, as will be discussed below in greater detail.

Examples of biomaterials 29 which may be used for disc augmentation can be natural or synthetic, resorbable or non-resorbable. Natural materials include various forms of collagen that are derived from collagen-rich or connective tissues such as an intervertebral disc, fascia, ligament, tendon, skin, demineralized bone matrix, etc. Material sources include autograft, allograft, xenograft, human-recombinant origin, etc. Natural materials also include various forms of polysaccharides that are derived from animals or vegetation such as hyaluronic acid, chitosan, cellulose, agar, etc. Other natural materials include other proteins such as fibrin, albumin, silk, elastin and keratin. Synthetic materials include various implantable polymers or hydrogels such as silicone, polyurethane, silicone-polyurethane copolymers, polyolefin, polyester, polyacrylamide, polyacrylic acid, polyvinyl alcohol, polyethylene oxide, polyethylene glycol, polylactide, polyglycolide, poly(lactide-co-glycolide), poly(dioxanone), poly(ε-caprolactone), poly(hydroxylbutyrate), poly(hydroxylvalerate), tyrosine-based polycarbonate, polypropylene fumarate or combinations thereof. It is preferred that the biomaterial can undergo transition from a flowable to a non-flowable state shortly after injection. This can typically be achieved by adding a crosslinking agent to the biomaterial before, during, or after injection.

Proteoglycans may also be included in the injectable biomaterial 29 to attract and/or bind water to keep the disc nucleus 18 hydrated. Similarly, growth factors (e.g. transforming growth factor beta, bone morphogenetic proteins, fibroblast growth factors, platelet-derived growth factors, insulin-like growth factors, etc.)_and/or other cells (e.g., intervertebral disc cells, stem cells, etc.) to promote healing, repair, regeneration and/or restoration of the disc, and/or to facilitate proper disc function, may also be included. Additives appropriate for use in the claimed invention are known to persons skilled in the art, and may be selected without undue experimentation.

Injectable biomaterial 29 is preferably mixed with the radiographic contrast medium prior to injection into the disc nucleus 18. This will allow the injection to be monitored using fluoroscopy. The catheter 26 or the needle 30, FIG. 2F, used for injection, is removed after an appropriate volume of biomaterial is deposited in the disc nucleus 18.

As an alternative to withdrawing the balloon 28, as illustrated in FIG. 2D above, a balloon 128, FIG. 3 may be detachable at 127 from a catheter 126, and may remain inflated in the disc nucleus 18 as an implant. In the case of the detachable balloon 128, it may be advantageous to inject a biomaterial which, after injection, takes a set in an elastic or gel form. This could be accomplished by injecting a second material with the biomaterial which would alter the form of the injected material.

As an alternative to inflating balloon 28 with the radiographic contrast medium as described above, the balloon 28 may be inflated by injection of the biomaterial 29. This would be advantageous in the embodiment described above where the balloon is detachable and where the biomaterial may take a set after injection.

In the case of direct injection of biomaterial 29 into the inflatable balloon member 28, the balloon 28 may be porous or permeable (e.g. woven fabric, mesh structure, perforated membrane, etc.) to allow material or fluid migration out of the inflatable member during or after injection.

Alternatively, a modified balloon 28a, FIG. 4, may be of a shape including a profiler for inflating in a pattern for spreading the endplates 22 apart. That is, the balloon 28a is manufactured to expand to a suitable shape to better accomplish spreading the endplates 22 apart rather than to conform to the shape of disc nucleus 18 as in FIG. 2C.

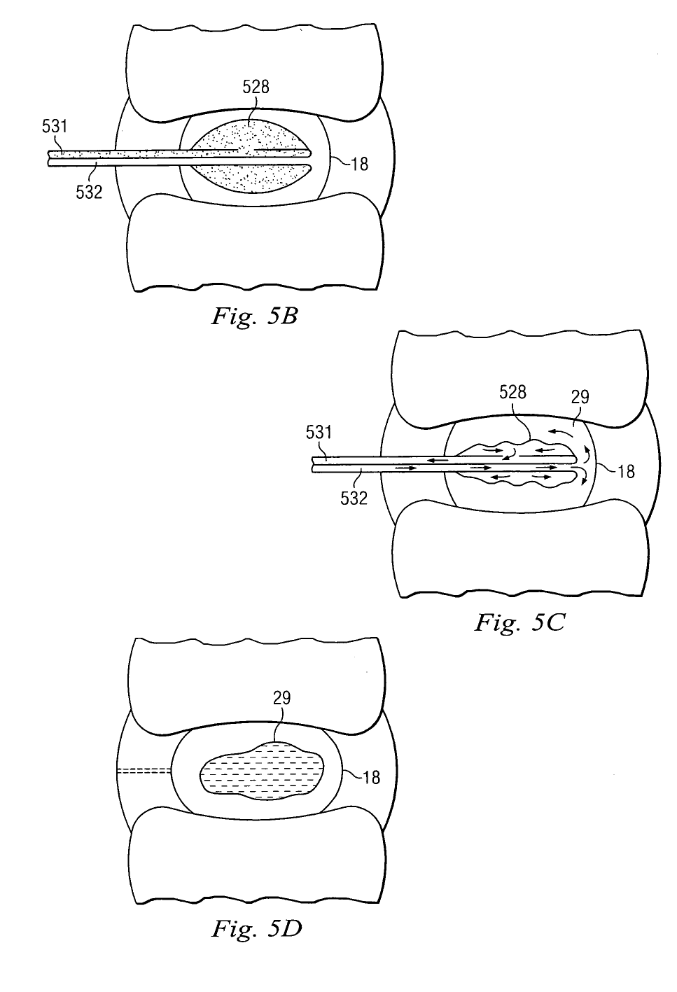

An alternative balloon catheter may be used, i.e. a double lumen catheter which can be used for injection as the balloon is being deflated. In an alternative embodiment, FIG. 5A illustrates a balloon catheter 526 introduced into the disc nucleus 18. The catheter 526 includes a first channel 531, a second channel 532 and a balloon 528. The saline and/or radiographic contrast medium is injected into balloon 528 via the first channel 531 to inflate balloon for expansion of the disc nucleus 18. In FIG. 5B, the inflated balloon 528 remains inflated in the disc nucleus 18 for an appropriate amount of time to stretch the annulus fibrosis and/or expand the nuclear disc space. In FIG. 5C, an appropriate biomaterial 29 is injected into the disc nucleus 18 via the second channel 532 in catheter 526 while the balloon inflating medium is simultaneously evacuated via the first channel 531. In FIG. 5D, the deflated balloon 528 is withdrawn with catheter 526 from the disc nucleus 18 and the injected biomaterial 29 remains within the disc nucleus 18.

As a result, one embodiment provides an apparatus including a high-pressure balloon catheter with a small shaft diameter (3 mm or smaller, preferably 2 mm or smaller, most preferably 1 mm or smaller). The catheter has a pointed tip for puncturing an intact disc annulus and insertion of the balloon section into the nuclear disc region. The catheter either has rigid shaft or is supported by a rigid guide-needle during penetration into the disc. For a rigid shaft, the catheter can be made of metal tubing. For a flexible shaft, the catheter can be made of polymeric tubing and is supported with a rigid guide-needle or guide-wire. If a guide-needle is used, the catheter can be double lumen. The balloon has an appropriate final volume of from about 0.1 cc to about 8.0 cc, preferably up to 5.0 cc and dimensions (length=5-40 mm, preferably 10-30 mm; diameter=3-20 mm, preferably 5-15 mm) to fit the nuclear disc region. The balloon can be of various shapes; conical, spherical, square, long conical, long spherical, long square, tapered, stepped, dog bone, offset, or combinations thereof. Balloons can be made of various polymeric materials such as polyethylene terephthalates, polyolefins, polyurethanes, nylon, polyvinyl chloride, silicone, polyetheretherketone, polylactide, polyglycolide, poly(lactide-co-glycolide), poly(dioxanone), poly(ε-caprolactone), poly(hydroxylbutyrate), poly(hydroxylvalerate), tyrosine-based polycarbonate, polypropylene fumarate or combinations thereof.

Another embodiment provides first, a determination that the treated disc has a competent and intact annulus fibrosis for safe expansion and effective containment of the subsequently injected biomaterial. After the annulus quality and integrity are verified using discography, the disc expansion device with the smallest shaft diameter possible, is inserted into the center of the disc. Insertion of the device can be done percutaneously, preferably under fluoroscopic guidance. The balloon is gradually inflated with radio-contrast fluid or saline to pressurize the disc, and thereby, stretch the annulus fibrosis. After a predetermined inflation time, the balloon is deflated and removed from the disc space. The biomaterial is subsequently injected into the disc using a small-diameter hypodermic needle until a desirable injection volume is achieved. When a double-lumen catheter is employed, the biomaterial can be injected into the disc through the same catheter during or after balloon deflation. The whole procedure is preferably done under fluoroscopic guidance.

The foregoing has described an apparatus and method for expansion of an intervertebral disc prior to its augmentation with an injectable biomaterial. Disc expansion prepares the disc annulus to receive a desirable or effective volume of injectable material in a single treatment. Because the annulus fibrosis is a viscoelastic material, it can be temporarily stretched as the disc is expanded under pressure.

Although illustrative embodiments have been shown and described, a wide range of modification, change and substitution is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

What is claimed is:

1. A method of treating an intervertebral disc of a patient, the method comprising the steps of:
inserting a balloon into a nucleus pulposus without removing material from the nucleus pulposus, the balloon being connected to a catheter;
inflating the balloon with a biomaterial; and
removing the catheter from the patient while leaving the inflated balloon in the patient.

2. The method of claim 1, wherein inflating the balloon comprises introducing the biomaterial through the catheter.

3. The method of claim 1, further comprising:
providing the biomaterial as a formulation that includes one or more types of cells effective to promote healing, repair, regeneration and/or restoration of the intervertebral disc.

4. The method of claim 1, further comprising:
verifying the location of the balloon within the nucleus pulposus.

5. The method of claim 4, wherein the location of the balloon is verified by fluoroscopy.

6. The method of claim 5, wherein the inflation of the balloon is monitored by fluoroscopy.

7. The method of claim 6, wherein the biomaterial is at least partially radiopaque.

8. A method of augmenting an intervertebral space, the method comprising the steps of:
inserting a balloon into a nucleus pulposus without removing material from nucleus pulposus, the balloon being connected to a catheter;
inflating the balloon with a first material;
allowing the balloon to remain inflated for a period of time;
removing the first material from the balloon;
injecting into the balloon a second material, the second material comprising a biomaterial; and
removing the catheter from the patient while leaving the balloon injected with the second material in the patient.

9. The method of claim 8, wherein inflating the balloon comprises introducing the first material through the catheter.

10. The method of claim 9, wherein removing the first material comprises extracting the first material through the catheter and wherein injecting the second material comprises introducing the second material through the catheter.

11. The method of claim 10, wherein removing the first material and injecting the second material are performed simultaneously.

12. The method of claim 11, wherein the catheter comprises a dual-lumen catheter and the first material is removed through a first lumen of the catheter and the second material is injected through a second lumen of the catheter.

13. The method of claim 12, wherein the balloon has an inflated volume from about 0.1 cc to about 8.0 cc.

14. The method of claim 13, wherein the balloon has an inflated volume sized to at least partially distract the vertebrae adjacent to the nucleus pulposus.

15. A method of treating a patient, comprising the steps of:
inserting a balloon into a nucleus pulposus of an intervertebral disc without removing material from nucleus pulposus, the balloon being connected to a catheter;
inflating the balloon with a first material through the catheter;
allowing the balloon to remain inflated within the nucleus pulposus for a period of time;
removing the first material from the balloon leaving a prepared space within the nucleus pulposus;
removing the balloon from the patient; and
injecting a biomaterial into the prepared space of the nucleus pulposus.

16. The method of claim 15, wherein the biomaterial is selected from the group consisting of collagen, fibrin, albumin, silk, elastin, keratin, and other proteoglycans.

17. The method of claim 16, wherein the biomaterial further comprises a polysaccharide selected from the group consisting of hyaluronic acid, chitosan, cellulose, and agar.

18. The method of claim 15, wherein the biomaterial includes a synthetic material.

19. The method of claim 18, wherein the synthetic material includes an elastomeric material from a group consisting of silicone, polyurethane, silicone-polyurethane copolymers, polyolefin, and combinations thereof.

20. The method of claim 19, wherein the synthetic material includes a hydrogel material from a group consisting of polyester, polyacrylamide, polyacrylic acid, polyvinyl alcohol, polyethylene oxide, polyethylene glycol, and combinations thereof.

21. The method of claim 19, wherein the synthetic material includes a resorbable polymer material from a group consisting of polylactide, polyglycolide, poly(lactide-co-glycolide), poly(dioxanone), poly(ε-caprolactone), poly(hydroxylbutyrate), poly(hydroxylvalerate), tyrosine-based polycarbonate, polypropylene fumarate, and combinations thereof.

22. A method treating an intervertebral disc of a patient, the method comprising:
forming and dilating an opening in a disc annulus without removing any of the disc annulus;
introducing an inflatable member through the dilated opening in the disc annulus and into a nucleus pulposus of the disc without removing any of the nucleus pulposus;
inflating the inflatable member with a first material to augment a space in the nucleus pulposus;
monitoring the internal pressure and expansion of the inflatable member;
extracting the first material from the inflatable member; and
injecting a biomaterial into the inflatable member.

23. The method of claim 22, wherein injecting the biomaterial comprises injecting the biomaterial through a passage in the inflatable member simultaneously with the extraction of the first material.

24. The method of claim 23, wherein the first material is extracted through a first lumen of a dual-lumen catheter and the biomaterial is injected through a second lumen of the dual-lumen catheter.
